(19) United States
(12) Patent Application Publication
(10) Pub. No.: US 2002/0102259 A1
(43) Pub. Date: Aug. 1, 2002

(54) Methods and compositions for stimulating CD 45 and thereby suppressing microglial activation associated with Alzheimer's disease

(76) Inventors: Jun Tan, Tampa, FL (US); Terrence Town, Tampa, FL (US); Michael Mullan, Tampa, FL (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 09/985,598

(22) Filed: Nov. 5, 2001

Related U.S. Application Data
(60) Provisional application No. 60/245,608, filed on Nov. 3, 2000.

(51) Int. Cl. A61K 39/395

(52) U.S. Cl. 424/144.1

(57) ABSTRACT

A method of inhibiting the negative effects of beta-amyloid in the brain of an animal comprising administering an effective amount of a compound that modulates CD45 activity. This invention also relates to compositions for use in stimulating CD45, and assays for use in finding compounds useful in inhibiting the negative effects of beta-amyloid. An assay for use in identifying compounds that inhibit the negative effects of beta-amyloid comprises (a) contacting immune cells with a predetermined amount of one or more test compounds in the presence of beta-amyloid or one or more peptides derived therefrom, (b) monitoring the amount of one or more inflammatory molecules released by the immune cells, and (c) comparing the amount found in step (b) with another amount found in the absence of the one or more test compounds or using a different predetermined amount the one or more test compounds.

### FIELD OF THE INVENTION

This invention relates generally to methods and compositions for use in the treatment of Alzheimer's and related amyloidogenic diseases, and to methods for screening such compounds. More specifically, this invention relates to β-amyloid peptide-induced microglial activation. This invention relates more particularly to compounds that stimulate CD45, a membrane-bound protein tyrosine phosphatase (PTP), thereby reducing microglial activation and other Alzheimer pathologies such as amyloid deposition and/or tau pathologies, and to animal and cell-based methods for identifying such compounds.

### BACKGROUND OF THE INVENTION

Alzheimer's disease (AD) is the most common progressive dementing illness, and is neuropathologically characterized by deposition of the 40 to 42 amino acid β amyloid peptide (Aβ) (proteolytically derived from the amyloid precursor protein, APP) as senile plaques. Concomitant with Aβ deposition there exists robust activation of inflammatory pathways in AD brain, including production of pro-inflammatory cytokines and acute-phase reactants in and around Aβ deposits (McGeer and McGeer, 1999; McGeer and McGeer, 1998; Rogers et al., 1996). Activation of the brain's resident innate immune cells, the microglia, is thought to be intimately involved in this inflammatory cascade, as reactive microglia produce pro-inflammatory cytokines such as tumor necrosis factor alpha (TNF-α) and interleukin-1β, which (at high levels) promote neurodegeneration (Rogers et al., 1996; Meda et al., 1995; Barger and Harmon, 1997). In support of the hypothesis that microglial activation leading to inflammation is detrimental, epidemiological studies have shown that patients using non-steroidal anti-inflammatory drugs (NSAIDs) have as much as 50% reduced risk for AD (Rogers et al., 1996; Stewart et al., 1997), and post-mortem evaluation of AD patients who underwent NSAID treatment has demonstrated that risk reduction is associated with diminished numbers of activated microglia (Mackenzie and Munoz, 1998). Further, when transgenic mice that overexpress the “Swedish” APP mutation (Tg APP_{SW}) are given the NSAID ibuprofen, these animals show reduction in Aβ deposits, astrocytosis, and dystrophic neurites correlating with decreased microglial activation (Lim et al., 2000).

Microglial activation has been implicated as pathogenic in a variety of neurodegenerative diseases in addition to AD, such as multiple sclerosis and AIDS dementia, raising the possibility that therapeutic strategies aimed at opposing microglial activation may be beneficial in treating such diseases. It believed that secretion of cytokines by activated microglia plays a key role in the inflammatory processes of neurodegenerative diseases such as Alzheimer's Disease (AD) because cytokines such as TNF-α and IL-1β promote neurodegeneration (Meda et al., 1995; Rogers et al., 1996; Barger and Harmon, 1997). There is mounting evidence that products of the inflammatory process in AD brain exacerbate AD pathology. Many of these inflammatory proteins and acute phase reactants such as alpha 1 antichymotrypsin, transforming growth factor β, apolipoprotein E and complement factors are produced by activated glia, are localized to Aβ plaques, and have been shown to promote Aβ plaque “condensation” or maturation (Nilsson et al., 2001; Harris-White et al., 1998; Styren et al., 1998; Rozemuller et al., 1989). Further, there is evidence that activated microglia in AD brain, instead of clearing Aβ, are pathogenic by promoting Aβ fibrillogenesis and consequent deposition as senile plaque (Frackowiak et al., 1992; Wegiel et al., 2000).

However, recent studies have indicated that the relationship between microglial activation and promotion of AD-like pathology is not straightforward, as some forms of microglial activation appear to mitigate this pathology. Schenk et al, have shown that immunization of the PDAPP mouse model of AD with Aβ₁₋₄₂ results in marked reduction of Aβ deposits, and atypical punctate structures containing Aβ that resembled activated microglia were found in brains of these mice, suggesting that immunization activates microglia to phagocytose Aβ (Schenk et al., 1999). This hypothesis was further supported ex vivo, where microglia were shown to clear deposited Aβ that was opsonized by anti-Aβ antibodies (Bard et al., 2000). Similar prophylactic effects of Aβ₁₋₄₂ immunization have now been independently observed in other transgenic mouse models of AD (Morgan et al., 2000; Janus et al., 2000), and in vivo visualization has shown that application of anti-Aβ antibody to PDAPP mouse brain results in rapid Aβ plaque clearance associated with marked local microglial activation (as measured by lectin immunoreactivity) (Bacskai et al., 2000). Finally, bigenic mice that overexpress human APP and transforming growth factor β1 also demonstrate reduced parenchymal Aβ deposition associated with an increase in microglia positive for the F4/80 antigen (Wyss-Coray et al., 2001). Tg APP_{SW} mice manifest prominent astrocytosis and microgliosis and develop amyloid deposits comparable to human senile plaques by 16 months of age (Irizarry et al., 1997).

Current attempts at reducing neuroinflammation mediated via microglial activation have only been partially efficacious (Rich et al., 1995), possibly owing to the fact that such strategies are more general inhibitors of inflammation than specific inhibitors of microglial-associated neuroinflammation. For example, Mackenzie and Munoz (1998) examined postmortem brain tissue from AD patients who underwent NSAID treatment and control individuals who did not use NSAIDs (Mackenzie et al., 1998). These authors found that while there were no significant differences in the mean numbers of senile plaques, senile plaque subtypes (diffuse or neuritic) or neurofibrillary pathology between cases and controls, the numbers of activated microglia were significantly decreased in NSAID-treated AD patients compared to controls. These data suggest that NSAIDs are prophylactic for AD partly by virtue of their opposition of microglial activation. However, current anti-inflammatory therapeutics directed against AD, such as non-steroidal anti-inflammatory drugs (NSAIDs), only partially suppress microglial activation (Mackenzie and Munoz, 1998), and, therefore, may not provide the greatest therapeutic benefits for AD. This suggestion is supported by clinical evidence, where elderly persons using NSAIDs demonstrate only an approximate 20% reduction in risk for AD (Beard et al., 1998), and AD patients using NSAIDs enjoy only a partial amelioration of disease symptoms (Rich et al., 1995). Thus, a more viable therapeutic strategy may be combination of NSAIDs with specific inhibitors of microglial activation. Following from this idea, pharmacotherapeutics specifically aimed at blocking microglial activation may well be more efficient at ameliorating microglial-associated neuropathology in AD.

Knowledge concerning the molecular mediators of microglial activation comes from studies involving peripheral lymphocytes. For example, the CD40-CD40L signaling pathway is involved in both T cell and microglial activation (Yang and Wilson, 1996; Tan et al., 1999a; Maxwell et al., 1999), yet, while blockade of this pathway has proved an efficient means of opposing T cell activation (Grewal et al., 1996; Stuber et al., 1996), interruption of this pathway is largely unexplored as a means of opposing microglial activation. Indeed, Applicants recently demonstrated that ligation of microglial CD40 synergistically enhances activation of these cells by a low dose of freshly solubilized Aβ (Tan et al., 1999b), indicating that the CD40-CD40L interaction is critically involved in microglial activation-induced Aβ.

In searching for novel cell surface molecules that may play a role in opposing microglial activation, Applicants focused on CD45, a functional transmembrane protein tyrosine phosphatase (PTP) that, when cross-linked, has been shown to play a critical role in negative regulation of T and B lymphocyte activation (Justement, 1996). CD45 is a key immunoregulatory molecule, and we have previously shown that CD45 opposes Aβ peptide-induced microglial activation by inhibiting p44/42 Mitogen-Activated Protein Kinase (MAPK). CD45 was selected because microglia express it in the frontal cortex and hippocampus of normal aging individuals, and this expression level is markedly increased in these brain regions in AD cases (Masliah et al., 1991; Licastro et al., 1998). Furthermore, in an animal model of neurodegeneration, upregulation of phosphotyrosine signal associated with activated microglia was found in and around the degenerating brain region (Karp et al., 1994). These data led Applicants to investigate the possible involvement of CD45 PTP signaling as a putative regulator of microglial activation.

It has previously been shown that CD45 and the TNF receptor superfamily member CD40 can antagonize each another, as stimulation of CD45 opposes CD40-induced Ig class switching of human B cells to the IgE isotype (Loh, et al., 1995). The mechanism underlying CD45/CD40 antagonism involves dephosphorylation/phosphorylation of tyrosine residues on their respective target signaling proteins, as CD45 is a PTP and ligation of CD40 results in protein tyrosine phosphorylation (Lazaar et al., 1998; Friedman et al., 1999). We have recently shown that ligation of CD40 leads to activation of microglia, as evidenced by TNF-α release and bystander-induced neurotoxicity (Tan et al., 1999a).

None of the above compositions or methods address the problem of treating AD by administering a composition that stimulates CD45, thereby suppressing microglial activation, or address the need for an assay for finding compounds useful in treating AD in this manner. In view of the complex nature of Alzheimer's disease, and the present lack of effective means for treating and preventing it, there is a need in the art for compositions and methods of treating AD, and assays for discovering compounds that are useful in treating AD.

### SUMMARY OF THE INVENTION

The methods, compositions, and assays of this invention address the need in the art for an effective treatment for AD and other neurodegenerative disorders, as set forth above. More specifically, and according to a preferred aspect of this invention, a method of inhibiting the negative effects of beta-amyloid in the brain of an animal comprises administering an effective amount of a compound that modulates CD45 activity.

According to an additional aspect of this invention, an assay for compounds that inhibit the negative effects of beta-amyloid comprises (a) contacting immune cells with a predetermined amount of one or more test compounds in the presence of one or more β-amyloid (Aβ) peptides, (b) monitoring the amount of one or more inflammatory molecules released by the immune cells, and (c) comparing the amount found in step (b) with another amount found (i) in the absence of the one or more test compounds or (ii) using a different predetermined amount of the one or more test compounds.

According to yet another aspect of this invention, an assay for compounds that inhibit the negative effects of beta-amyloid comprises (a) contacting β-amyloid overproducing immune cells with a predetermined amount of one or more test compounds in the presence of one or more stimulatory molecules, (b) monitoring the amount of one or more metabolites of amyloid precursor protein (APP) produced by the β-amyloid overproducing immune cells, and (c) comparing the amount found in step (b) with another amount (i) found in the absence of the one or more test compounds or (ii) using a different predetermined amount of the one or more test compounds.

According to a further aspect of this invention, an assay for identifying compounds that bind to CD45 comprises contacting CD45 with a predetermined amount of one or more test compounds in the presence of a substrate of CD45 and measuring the amount of phosphorylation of the substrate.

According to yet another aspect of this invention, an in vivo assay for determining the biological activity of a test compound comprises (a) crossing a first animal model of Alzheimer's disease with a second animal deficient in CD45, (b) administering a predetermined amount of one or more test compounds to the offspring of step (a), and (c) determining the effects of β-amyloid in the brain of the offspring.

According to a further aspect of this invention, an assay comprises (a) obtaining or generating an animal model of neurodegenerative disease, (b) administering a molecule that modulates CD45 activity to the animal model, and (c) measuring negative effects of neurodegeneration in the animal model.

It will be apparent to those skilled in the art that only the preferred embodiments have been described by way of exemplification, and that there are various possible modifications that fall within the scope of this invention. These and other aspects of the invention will be discussed in greater detail below.

### BRIEF DESCRIPTION OF THE FIGURES

Other advantages of the present invention will be readily appreciated as the same becomes better understood by reference to the following detailed description when considered in connection with the accompanying drawings, wherein:

FIGS. 1(a), (b), and (c) show that co-treatment with phen and Aβ peptides synergistically affects microglial activation. Microglial cells were treated as indicated for 12 hours, or co-cultured with primary neuronal cells (microglia:neurons, 1:2) under the same indicated treatment conditions for 48 hours. Control peptide is Aβ₄₀₋₁. Microglial activation was measured by (a) TNF-α production (mean+−1 SEM, pg/mg total protein) in cultured media by TNF-α ELISA, (b) NO release (mean±1 SEM, mM/mg total protein) in cultured media by NO assay, and (c) Neuronal cell injury by LDH assay (mean LDH (%) release±1 SEM). Data shown in (a) and (b) are representative of five independent experiments, and data in (c) are representative of two independent experiments. For (a), (b), and the neuronal/microglial co-culture conditions in (c), ANOVA revealed significant main effects of Aβ₁₋₄₀(p<0.001), Aβ₁₋₄₂ (P<0.001), and phen (p<0.001). There were also significant interactions between Aβ₁₋₄₀ or Aβ₁₋₄₂ and phen (p<0.001). One-way ANOVA revealed significant inter-group differences (p<0.001), and post hoc testing revealed significant differences between phen/Aβ₁₋₄₀ or phen/Aβ₁₋₄₂ when compared to phen/control peptide (p<0.001).

FIGS. 2(a), (b), and (c) illustrate that CD45 cross-linking markedly inhibits phen and Aβ peptide-induced microglial activation. Microglial cells were treated as indicated for 12 hours, or co-cultured with primary cultured neuronal cells (microglia:neurons, 1:2) under the same treatment conditions. Control antibody is rat IgG2b. Microglial activation was determined by (a) TNF-α production (mean±1 SEM, pg/mg total protein) in cultured media, (b) NO release (mean±1 SEM, mM/mg total protein) in cultured media, and (c) neuronal cell injury (mean LDH (%) release±1 SEM) in co-culture experiments. Data shown in (a) and (b) are representative of five independent experiments, and data in (c) are representative of two independent experiments. For (a) and (b), one-way ANOVA revealed significant inter-group differences (p<0.001), and post hoc testing revealed significant differences between phen/Aβ₁₋₄₀/anti-CD45 and phen/Aβ₁₋₄₀/control antibody (p<0.01) or phen/Aβ₁₋₄₂/anti-CD45 and phen/Aβ₁₋₄₂/Control antibody (p<0.02). For the neuronal/microglial co-culture conditions in (c), one-way ANOVA revealed significant inter-group differences (p<0.001), and post hoc testing revealed a significant difference between phen/Aβ₁₋₄₂/anti-CD45 and phen/Aβ₁₋₄₂/control antibody (p<0.02).

FIGS. 3(a), (b), (c), (d), (e), and (f) show that co-treatment of microglia with phen and Aβ peptides activates p44/42 MAPK resulting in microglial activation. Microglial treatment conditions are indicated, and are further described in methods. Control peptide is Aβ₄₀₋₄. Cell lysates were analyzed by Western immunoblotting using specific antibodies which recognize phosphorylated or total p44/42 MAPK; (a) and (b) show phosphorylation and activity of p44/42 MAPK following co-treatment with phen and Aβ peptides; (c) and (d) show inhibition of this effect by PD98059 (a specific MEK1/2 inhibitor). Histograms below immunoblots represent the mean band density ratio±1 SEM (phospho-p42 MAPK/total p42 MAPK) and band density in ODm (phospho-Elk 1), respectively (n=3 for each condition presented). Microglial activation is evidenced by (e), mean TNF-α release±1 SEM (pg/mg total protein) and, (f), mean NO release±1 SEM (mM/mg total protein) in cultured media by ELISA or by NO release assay, respectively (n=3 for each condition presented). For (a) and (b), ANOVA revealed significant main effects of phen, Aβ₁₋₄₀, and Aβ₁₋₄₂ (P<0.001), and there were significant interactive terms between phen and either Aβ₁₋₄₀ or Aβ₁₋₄₂ (P<‘001)’ One-way ANOVA revealed significant inter-group differences (p<0.001), and post hoc testing showed significant differences between Aβ₁₋₄₀ and phen/Aβ₁₋₄₀ (P<0.001) and between Aβ₁₋₄₀ and phen/Aβ₁₋₄₀ (P<0.001). For (c)-(f), one-way ANOVA revealed significant inter-group differences (p<0.001), and post hoc testing showed significant differences between phen/Aβ₁₋₄₀ , and phen/Aβ₁₋₄₀ /PD98059 (p<0.01), and between phen/Aβ₁₋₄₀ and phen/Aβ₁₋₄₂/PD98059 (p<0.01).

FIGS. 4(a) and (b) illustrate that cross-linking of microglial CD45 markedly suppresses p44/42 MAPK activation resulting from phen and Aβ peptide co-treatment. Microglial treatment conditions are indicated, and are further described in methods. Cell lysates were analyzed by Western immunoblotting using specific antibodies which recognize (a) phosphorylated or total p44/22 MAPK, or (b) the p44/22 MAPK fusion protein, Elk-1, by immune complex kinase assay. Histograms below immunoblots represent (a) the mean band density ratio±1 SEM (phospho-p42 MAPK/total p42 MAPK) or (b), mean band density±1 SEM in ODm, with n=3 for each condition presented. For (a) and (b), ANOVA revealed significant main effects of Aβ₁₋₄₀, and Aβ₁₋₄₂ (P<0.001), and there was statistical interaction between either Aβ₁₋₄₀ or Aβ₁₋₄₂ and phen (p<0.001). One way ANOVA revealed significant inter-group differences (p<0.001), and post hoc testing showed significant differences between phen/Aβ₁₋₄₀ and phen/Aβ₁₋₄₂/anti-CD45 (p<0.001), and between phen/Aβ₁₋₄₂ and phen/Aβ₁₋₄₂/anti-CD45 (p<0.001).

FIGS. 5(a), (b), and (c) show that stimulation of CD45-deficient microglia with Aβ peptides results in microglial activation. Primary cultured wild-type or CD45-deficient microglial cells were treated as indicated for 12 hours, or co-cultured with primary cultured neuronal cells under the same treatment conditions for 36 hours. Control peptide is Aβ₁₋₄₀. Microglial activation is evidenced by (a) TNF-α production (mean 1 SEM, n=3 for each condition presented), (b) NO assay (mean±1 SEM, n 3 for each treatment condition), and (c) neuronal cell injury in co-culture experiments (mean LDH (%) release±1 SEM, n=3 for each condition presented). For CD45-deficient microglia in (a) and (b), one way ANOVA revealed significant inter-group differences (p<0.001), and post hoc testing showed significant differences between control peptide and either Aβ₁₋₄₀ (p<0.001) or Aβ₁₋₄₂ (p<0.001). For neuronal/CD45 microglial co-culture experiments, one-way ANOVA revealed significant inter-group differences (p<0.001), and post-hoc testing showed significant differences between control peptide and either Aβ₁₋₄₀ (p<0.001) or Aβ₁₋₄₂ (P<0.001).

FIG. 6 shows that TNF-α production in vivo is markedly increased in Tg APP_{SW} mice deficient for CD45. Brains from 6-month-old transgenic mice were isolated and prepared for TNF-α Western immunoblotting as described in Methods. TNF-α protein immunoblots (above) and mean band density ratios to actin±1 SEM (below, n=3 for each condition) are shown. ANOVA revealed significant main effects of CD45 deficiency (CD45 def., p<0.001) and Tg APP_{SW} status (p<0.001), as well as a significant interactive term between them (p<0.01). One way ANOVA revealed significant inter-group differences (p<0.001), and post hoc testing showed significant differences between control and Tg APP_{SW} (p<0.01) or CD45 def. (p<0.05), and between either Tg APP_{SW} or CD45 def. and the crossed mice (p<0.001).

### DETAILED DESCRIPTION OF THE INVENTION

While this invention will be described primarily with respect to methods for inhibiting the negative effects of Aβ, and an assay for identifying compounds useful in inhibiting the negative effects of Aβ, it is to be understood that the features thereof will find applicability to other applications, such as the treatment of AD, as well as other neurodegenerative disorder. The term “CD45” as used herein is meant to refer to native, recombinant or synthetic forms of the molecule.

Briefly, this invention relates to methods of inhibiting the negative effects of Aβ, compositions for inhibiting the negative effects of Aβ, and an assay for identifying compounds useful in inhibiting those negative effects. While the embodiments of the invention exemplified herein are directed to Alzheimer's Disease (AD), the present invention is contemplated to also include related “amyloidogenic diseases,” including but not limited to scrape; transmissible spongioform encephalopathies (TSEs); hereditary cerebral hemorrhage with amyloidosis, Icelandic-type (HCHWA-I); hereditary cerebral hemorrhage with amyloidosis, Dutch-type (HCHWA-D); Familial Mediterranean Fever; Familial amyloid nephropathy with urticaria and deafness (Muckle-Wells syndrome); myeloma or macroglobulinernia-associated idopathy associated with amyloid; Familial amyloid polyneuropathy (Portuguese); Familial amyloid cardiomyopathy (Danish); Systemic senile amyloidosis; Familial amyloid polyneuropathy (Iowa); Familial amyloidosis (Finnish); Gerstmann-Staussler-Scheinker syndrome; Medullary carcinoma of thyroid; Isolated atrial amyloid; prion disease; Lewy body disease; frontotemporal dementia (tauopathies); Parkinson's disease; cerebrovascular disease; Islets of Langerhans; Diabetes type II; and Insulinoma, or combinations thereof. Many of these conditions are associated with deposition of amyloid plaques.

Reactive microglia have been suggested to play a role in the Alzheimer's disease (AD) process, and previous studies have shown that expression of CD45, a membrane-bound protein tyrosine phosphatase (PTP), is elevated in microglia in AD brains compared to controls. CD45 may also be expressed by other immune cells in the brain or elsewhere in the body. To investigate the possible role of CD45 in microglial responsiveness to β-amyloid (Aβ) peptides, we first co-treated primary cultured microglia with a tyrosine phosphatase inhibitor (phen, 5 mM) and freshly solubilized Aβ peptides (1000 nM). Data show synergistic induction of microglial activation as evidenced by TNF-α production and nitric oxide (NO) release, both of which we show to be dependent on activation of p44/22 mitogen activated protein kinase (MAPK).

Other negative effects of Aβ may include glutamate release, release of free radicals, superoxide dismutase, nitric acid synthase, neuronal cell injury, in addition to TNF-α production and NO release. One or more of these negative effects may be exhibited. Additional negative effects contemplated according to this invention include chronic activation of inflammatory cells, free radical-mediated damage to cells or tissues, neuronal apoptosis, neuronal death, release of inflammatory molecules, amyloid deposition, increased amyloid levels, increased formation of soluble or insoluble aggregates of Aβ, neuronal injury, tau pathology, or combinations of these.

Cross-linking of microglial CD45 by anti-CD45 antibody markedly inhibits these effects via inhibition of p44/22 MAPK, suggesting that CD45 is a negative regulator of microglial activation. Accordingly, primary cultured microglia from CD45-deficient mice demonstrate hyper-responsiveness to Aβ, as evidenced by TNF-α release, NO production, and neuronal injury following stimulation with Aβ peptides. As a validation of these findings in vivo, brains from a transgenic mouse model of AD (Tg APP_{SW}) deficient for CD45 demonstrate markedly increased production of TNF-α compared to Tg APP_{SW} mice.

Taken together, these results suggest that therapeutic agents that stimulate the CD45 PTP signaling pathway may be effective in suppressing microglial activation associated with AD. The therapeutic agents according to this invention may be agonists that stimulate CD45 activity, antibodies that stimulate CD45 activity, or agonists that oppose CD40 ligation or its effects.

Decreased activation of p44/22 MAPK was also observed under these conditions, suggesting that CD45 cross-linking stimulated the CD45-associated PTP pathway, and that stimulation of this pathway negatively controls p44/22 MAPK activation. In accordance with this, co-treatment of Aβ and phen-activated microglia with PD98059, an inhibitor of MEK1/2 (the upstream activator of p44/22 MAPK), resulted in statistically interactive blockade of microglial activation. We found that microglia deficient for CD45 could be directly activated by Aβ peptides in vitro, and brains from Tg APP_{SW} mice deficient for CD45 demonstrated markedly increased TNF-α levels compared to Tg APP_{SW} or CD45 deficient mouse brains. These results suggest that stimulation of CD45 is a viable approach for down-regulating Aβ-induced microglial activation.

Past studies have shown that, in general, phosphatase activity decreases with aging and even more so in AD across various cell types (Gong et al., 1995; Pei et al., 1998). Specifically, it has been shown that peripheral T lymphocytes isolated from AD patients demonstrate decreased amounts of the CD45R isoform compared to age-matched non-demented control subjects (Ikeda et al., 1991). In order to determine if increasing CD45 activity could block microglial activation resulting from co-treatment with phen and Aβ, we activated wild-type microglia, added CD45 recombinant protein (20 U/mL) to these cells, and measured NO and TNF-α release. Results showed that release of NO and TNF-α were markedly decreased following addition of CD45 recombinant protein compared to appropriate controls. Interestingly, treatment of activated microglia with CD45 recombinant protein resulted in blockade of NO and TNF-α release to a similar extent as cross-linking CD45, further substantiating that CD45 cross-linking stimulates the CD45 PTP pathway. Data show that cross-linking CD45 markedly reduces microglial activation resulting from Aβ and phen co-treatment.

Although we initially showed that co-treatment with the PTP inhibitor phen and Aβ peptides results in microglial activation, as evidenced by increased NO and TNF-α release, the question arose as to whether this effect was dependent on PTP inhibition as opposed inhibition of other phosphatases. Thus, we co-incubated wild-type primary culture microglia with Aβ and either sodium orthovanadate (1 mM), another PTP inhibitor, or okadaic acid (50 nM), an inhibitor of protein phosphatase 2A, and measured NO and TNF-α release. While sodium orthovanadate treatment in conjunction with Aβ produced results similar to phen and Aβ peptide co-treatment (data not shown), NO and TNF-α were not detectable in the media of okadaic acid and Aβ co-treated microglia (no significant inter-group differences by one-way ANOVA). This result suggests that treatment of microglia with specific inhibitors of PTPs, as opposed to general phosphatase inhibitors, along with Aβ triggers microglial activation, further substantiating the specific effect of PTP stimulation via CD45 in opposing microglial activation induced by phen and Aβ peptides.

Our data thus far had focused on CD45-mediated down-regulation of microglial activation induced by co-treatment with phen and Aβ. These data raised the question of whether CD45 may reduce microglial activation induced by other stimuli, such as LPS. To address this possibility, we incubated microglia with LPS (1 ng/mL) and anti-CD45 antibody. Data showed that CD45 cross-linking markedly attenuated microglial activation as evidenced by NO and TNF-α release (by one-way ANOVA followed by post-hoc comparison, p<0.001). These data raise the possibility that stimulation of the CD45 pathway negatively controls microglial activation induced by various pro-inflammatory stimuli, and suggest that pharmacotherapeutics targeting stimulation of CD45 may be beneficial in suppressing microglial activation, which is a pathogenic component of a variety of neurodegenerative diseases.

We presently show that inhibition of microglial CD45 leads to activation of these cells whereas stimulation of microglial CD45 opposes this effect. Note that one method for measuring activity of CD45 is to measure phosphorylation of a CD45 substrate, or levels of inorganic phosphate production. A preferred CD45 substrate according to this invention is src, a member of the src family, mitogen-activated protein kinases, the c-Jun N-terminal kinase pathway, the c-Jun N-terminal kinase activating kinase/signal transducers, activators of transcription pathway, or combinations of these. In the presence of low doses of freshly solubilized Aβ, synergistic enhancement of microglial activation is observed either in the CD40 ligation (Tan et al., 1999b) or CD45 inhibition paradigms. When taken together, these observations suggest that, in microglia, CD40 and CD45 may have antagonistic effects on activation of these cells, whereby CD40 promotes and CD45 opposes it. Additionally, the possibility arises that Aβ is able to positively affect microglial activation via disruption of CD45/CD40 homeostasis.

In a preferred form of the invention, the methods of using transgenic animals described herein, or cells derived therefrom, or functional equivalents, is contemplated which is eventually useful for treating, preventing and/or inhibiting conditions associated with plaques occurring in a tissue of the central nervous system of said animals or a patient. In another form, the methods claimed are useful against a disease of the internal organs related to amyloid plaque formation, including plaques in the heart, liver, spleen, kidney, pancreas, brain, lungs and muscles.

This invention may provide for transgenic gene and polymorphic gene animal and cellular (cell lines) models as well as for knockout models. These models are constructed using standard methods known in the art and as set forth in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; and 4,736,866, as well as Burke and Olson (1991), Capecchi (1989), Davies et al. (1992), Dickinson et al. (1993), Duff and Lincoln (1995), Huxley et al. (1991), Jakobovits et al. (1993), Lamb et al. (1993), Pearson and Choi (1993), Rothstein (1991), Schedl et al. (1993), Strauss et al. (1993). Further, patent applications WO 94/23049, WO 93/14200, WO 94/06908, WO 94/28123 also provide information relevant to this embodiment.

Assays

In a preferred embodiment, this invention provides assays for identifying small molecules or other compounds that are capable of modulating CD45 ligand pathways. The assays according to this invention preferably inhibit the negative effects of Aβ. The assays can be performed in vitro using non-transformed cells, immortalized cell lines, or recombinant cell lines.

Specifically, the assays of this invention are designed in a such a way as to detect the presence of increased or decreased inflammation or Alzheimer's disease pathology upon treatment with a candidate compound. According to one embodiment, the assay is performed by contacting immune cells with a test compound in the presence of Aβ (or a peptide derived therefrom). The amounts of various inflammatory molecules released by the immune cells is measured, and then compared to the amount of inflammatory molecules released in the absence of the test compound, or in the presence of a different amount of the test compound. The inflammatory molecules may include one or more of TNF-α, IL-1b, IL-6, IL-12, or TNF-γ, or they may also include nitric oxide, glutamate, a free radical species, or combinations of these. Preferably, according to one aspect, the immune cells are taken from a human being, and according to another aspect, the immune cells are selected from microglia, T cells, granulocytes, macrophages, astrocytes, and monocytes. The immune cells may optionally be deficient in CD45.

According to another embodiment of this invention, an assay includes contacting Aβ overproducing cells with a test compound, in the presence of at least one stimulatory molecule. The amount of metabolite of APP produced by the cells is monitored, and compared to the amount of metabolite produced by cells in the absence of the test compound, or in the presence of a different amount of the test compound. Preferably, the Aβ overproducing cells are CD45 deficient. An additional assay is also provided for identifying compounds that bind CD45, where CD45 is contacted with at least one test compound in the presence of a CD45 substrate. The amount of phosphorylation of the substrate is measured, and may be compared to the amount of phosphorylation in the absence of the test compound, or where a different amount of the test compound is used.

A further assay according to another embodiment includes an animal model of a neurodegenerative disease, to which a molecule that modulates CD45 activity is administered. The negative effects of the neurodegeneration in the animal model are measured. The neurodegenerative disease may be Parkinson's disease, Lewy body dementia, traumatic brain injury, tauopathies, prion disease, vascular dementia, or a combination of these. The neurodegenerative disease is preferably Alzheimer's disease. The molecule that modulates CD45 activity is preferably an antibody that stimulates CD45 activity, or a pharmacological agent.

In vivo assays are also contemplated by this invention, to allow determination of the biological activity of compounds to be tested. The method includes the steps of crossing a first animal model of a neurodegenerative disorder, such as AD, with a second animal model that is CD45 deficient. Test compounds may be administered to the crossed offspring, and the effects of the test compound can be measured by determining the effect, if any, of Aβ in the brain. The effects will likely be negative, and can be assessed qualitatively or quantitatively, or both.

Compounds identified by this method have utility in stimulating CD45, reducing microglial activation, and reducing inflammation in vivo. These compounds are then further tested in the animal models disclosed and enabled herein to identify those compounds having the most potent in vivo effects. In addition, as described above with respect to small molecules having such activity, these molecules can serve as “lead compounds” for the further development of pharmaceuticals by, for example, subjecting the compounds to sequential modifications, molecular modeling, and other routine procedures employed in rational drug design.

This invention also contemplates therapeutic compounds, which are preferably provided in a pharmaceutically acceptable carrier or diluent. The compounds of this invention, which are preferably identified using the assay system described in this invention, are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

Formulations

In the method of this invention, the compositions of this invention can be administered in various ways. It should be noted that they can be administered as the compound, or as pharmaceutically acceptable salt thereof, and can be administered alone or as an active ingredient in combination with pharmaceutically acceptable carriers, diluents, adjuvants, and vehicles. The compounds can be administered orally, subcutaneously, or parenterally. Parenteral administration includes administration by the following routes: intravenous, intramuscular, interstitial, intra-arterial, subcutaneous, intraocular, intrasynovial, transepithelial, including transdermal, pulmonary via inhalation, opthalmic, sublingual and buccal, topical, including ophthalmic, dermal, ocular, rectal, and nasal inhalation via insufflation or nebulization. . Implants of the compounds are also useful. The patient being treated is preferably a warm-blooded animal, more preferably, a mammal, and most preferably, is a human being. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is important to note that the compounds utilized or identified in the present invention are not restricted to a particular compartment of the body: for example, it is not required for efficacy that antibodies administered in methods of the present invention be administered to the CNS, or that they be administered peripherally. Such compounds may exert beneficial activity in either or both compartments. However, the active compounds can be administered to the CNS parenterally or intraperitoneally, if desired. Solutions of the compound as a free base or a pharmaceutically acceptable salt can be prepared in water mixed with a suitable surfactant, such as hydroxypropylcellulose. Dispersions can also be prepared in glycerol, liquid polyethylene glycols, and mixtures thereof, and in oils. Under ordinary conditions of storage and use, these preparations can contain a preservative and/or antioxidants to prevent the growth of microorganisms or chemical degeneration.

It is noted that humans are treated generally longer than mice or other experimental animals exemplified herein, and that treatment has a length proportional to the length of the disease process and drug effectiveness. The doses may be single doses or multiple doses over a period of several days, but single doses are preferred.

The compounds are preferably orally administered, for example, with an inert diluent or with an assimilable edible carrier, they can be enclosed in hard or soft shell gelatin capsules, or they can be compressed into tablets. For oral therapeutic administration, the active compounds can be incorporated with an excipient and used in the form of ingestible tablets, buccal tablets, troches, capsules, sachets, lozenges, elixirs, suspensions, syrups, wafers, and the like. The pharmaceutical composition comprising the active compounds can be in the form of a powder or granule, a solution or suspension in an aqueous liquid or nonaqueous liquid, or in an oil-in-water or water-in-oil emulsion.

The tablets, troches, pills, capsules and the like can also contain, for example, a binder, such as gum tragacanth, acacia, corn starch or gelatin, excipients, such as dicalcium phosphate, a disintegrating agent, such as corn starch, potato starch, alginic acid and the like, a lubricant, such as magnesium stearate, and a sweetening agent, such as sucrose, lactose or saccharin, or a flavoring agent. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring. Any material used in preparing any dosage unit form should be pharmaceutically pure and substantially non-toxic. In addition, the active compound can be incorporated into sustained-release preparations and formulations.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to this invention, however, any vehicle, diluent, or additive used would have to be compatible with the compounds. Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and any of the other ingredients from those enumerated above. In the case of sterile powders for the preparation of sterile injectable solutions, the preferred methods of preparation are vacuum drying and the freeze drying technique.

The compounds utilized in the present invention can also be administered parenterally to the patient in the form of slow-release subcutaneous implants or targeted delivery systems such as monoclonal antibodies, vectored delivery, ionophoretic, polymer matrices, liposomes, and microspheres. Examples of delivery systems useful in the present invention include those set forth in U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In one embodiment, the compound of the present invention can be administered initially by intravenous injection to bring blood levels to a suitable level. The patient's levels are then maintained by an oral dosage form, although other forms of administration can be used, dependent upon the patient's condition and other factors known to those of skill in the art. The quantity to be administered will vary for the patient being treated and will vary from about 100 ng/kg of body weight to 100 mg/kg of body weight per day and preferably will be from 10 mg/kg to 100 mg/kg per day.

Pharmaceutical compositions which are suitable for administration to the nose or buccal cavity include powder, self-propelling and spray formulations, such as aerosols, atomizers and nebulizers.

The compositions of this invention can also optionally contain other therapeutically active compounds which are usually applied in the treatment of the diseases and disorders discussed herein. Treatments using the present compounds and other therapeutically active compounds can be administered simultaneously or in intervals.

Gene Therapy

The term gene therapy, as used herein, refers to the transfer of genetic material (e.g. DNA or RNA) of interest into a host to treat or prevent a genetic or acquired disease or condition phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide or peptide of therapeutic value. Alternatively, the genetic material of interest encodes a suicide gene. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically re-implanted cells have been shown to express the transfected genetic material in situ.

In in vivo gene therapy, target cells are not removed from the subject, rather the genetic material to be transferred is introduced into the cells of the recipient organism in situ, that is within the recipient. In an alternative embodiment, if the host gene is defective, the gene is repaired in situ [Culver, 1998]. These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by those skilled within the art.

### EXAMPLES

The methods for inhibiting negative effects associated with the presence of Aβ in the brain, and assays for identifying compounds useful in minimizing the negative effects of Aβ of this invention will be described in more detail in the following non-limiting examples.

General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659; and 5,272,057, and incorporated herein by reference. Polymerase chain reaction (PCR) was carried out generally as in PCR Protocols: A Guide To Methods And Applications, Academic Press, San Diego, Calif. (1990). In-situ (in-cell) PCR in combination with Flow Cytometry can be used for detection of cells containing specific DNA and mRNA sequences (Testoni et al, 1996, Blood 87:3822.)

General Methods in Immunology

Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W. H. Freeman and Co., New York (1980).

Immunoassays

In general, ELISAs are the preferred immunoassays employed to assess a specimen. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989.

Antibody Production

Antibodies may be either monoclonal, polyclonal or recombinant. Conveniently, the antibodies may be prepared against the immunogen or portion thereof for example a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W. H. Freeman and Co., 1992. Antibody fragments may also be prepared from the antibodies and include Fab, F(ab′)2, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

For producing recombinant antibody (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complimentary DNAs (CDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as well known in the art. (For a general discussion of conjugation of fluorescent or enzymatic moieties see Johnstone & Thrope, Immunochemistry in Practice, Blackwell Scientific Publications, Oxford, 1982.) The binding of antibodies to a solid support substrate is also well known in the art. (see for a general discussion Harlow & Lane Antibodies: A Laboratory Manual, Cold Spring Harbor Laboratory Publications, New York, 1988 and Borrebaeck, Antibody Engineering—A Practical Guide, W. H. Freeman and Co., 1992) The detectable moieties contemplated with the present invention can include, but are not limited to, fluorescent, metallic, enzymatic, and radioactive markers such as biotin, gold, ferritin, alkaline phosphatase, b-galactosidase, peroxidase, urease, fluorescein, rhodamine, tritium, C¹⁴ and iodination.

### Example 1

### Stimulation of CD45 PTP Activity Via CD45 Cross-Linking

Materials and Methods

Monoclonal antibodies (purified rat anti-mouse CD45, such as leukocyte common antigen, clone no. LY-5, and purified rat IgG_{2b} control antibodies; FITC-conjugated rat anti-mouse CD45 and FITC conjugated rat IgG_{2b} control antibodies) were purchased from PharMingen (San Diego, Calif.). To test whether CD45 cross-linkage could result in stimulation of CD45 PTP activity, we measured free inorganic phosphate (Pi) in microglial cell lysates treated in the presence or absence of anti-CD45 antibody, and found significantly higher levels of Pi in microglial cell lysates treated with CD45 antibody compared to untreated cells. Antibodies for phospho-p44/22 mitogen activated protein kinase (MAPK) (Thr202/Tyr204), and total p44/22 MAPK were obtained from NEB (Beverly, Mass.). Aβ peptides and control peptide (Aβ₁₋₄₀) were obtained from QCB (Hopkinton, Mass.). Human CD45 recombinant protein and PD98059 were obtained from CALBIOCHEM (La Jolla, Calif.), as well as the phosphatase inhibitors including phen, sodium orthovanadate, and okadaic acid. Bacterial lipopolysaccharide (LPS) was purchased from Sigma. Anti-mouse HRP conjugated IgG secondary antibody and Western blotting luminol reagent were obtained from Santa Cruz Biotechnology (Santa Cruz, Calif.). Hy-bond PVDF membranes were purchased from Bio-Rad Laboratories (Hercules, Calif.). Anti-mouse TNF-α polygonal antibody was obtained from R&D systems (Minneapolis, Minn.).

Murine Primary Cell Culture

Breeding pairs of BALB/c and CD45 deficient (C57BL/601aHsd-Ptprc) mice were purchased from Jackson Laboratory (Bar Harbor, Maine) and housed in the animal facility at the University of South Florida Health Science Center. Tg APP_{SW} mice are the 2576 line backcrossed to C57B6/SJL as previously described (Hsiao et al., 1995; Hsiao et al., 1996). Murine primary culture microglia were isolated from mouse cerebral cortices and were grown in RPMI medium supplemented with 5% fetal calf serum, 2 mM glutamine, 100 U/mL penicillin, 0.1 μg/mL streptomycin and 0.05 mM 2-mercaptoethanol according to previously described methods (Chao et al., 1992). Briefly, cerebral cortices from newborn mice (1-2 days old) were isolated under sterile conditions and were kept at 4 degrees Centigrade prior to mechanical dissociation. Cells were plated in 75 cm³ flasks and complete medium was added. Primary cultures were kept for 14 days so that only glial cells remained, and microglia were isolated by shaking flasks at 200 rpm in a LabLine TM Incubator-Shaker. More than 98% of these glial cells stained positive for Mac-1 (CD11b /CD18; Boehringer Mannheim Biochemicals, Indianapolis, Ind.). Mouse primary culture neuronal cells were prepared as previously described (Chao et al., 1992). Briefly, cerebral cortices were isolated from BALB/c mouse embryos, between 15 to 17 days in utero, and cortices were mechanically dissociated in trypsin (0.5 g/mL) following incubation for 15 min at 37° C. Cells were collected following centrifugation at 1200 rpm and resuspended in DMEM (GIBCO, Gaithersburg, Md.) supplemented with 10% fetal calf serum, 10% horse serum, uridine (33.6 mg/mL, Sigma Chemical-Co., St. Louis, Mo.) and fluorodeoxyuridine (1 3.6 mg/mL, Sigma) and plated in 24 well tissue-culture plates at 2.5×1 O′ cells/well after collagen-coating the plates. After 10 days in vitro, cells were passed in preparation for subsequent experiments. More than 96% of these cells stained positive for neurofilament L (using rabbit anti-human neurofilament L antibody, Serotec Ltd, UK, data not shown). To verify CD45 deficiency status, CD45 expression in microglia isolated from CD45 -deficient mice was determined by FACS (fluorescence activated cell sorter) analysis as previously described (Tan et al., 1999a), and CD45 was undetectable in these cells (data not shown).

TNF-a ELISA and Nitric Oxide Release Assay

Primary cultured microglial cells were plated in 24-well tissue-culture plates (Costar, Cambridge, Mass.) at 5×10⁴ cells/well and stimulated for 12 h with phen (5 mM), Aβ peptides (1000 nM) or phen/Aβ peptides in the presence or absence of anti-CD45 antibody (1:200) or PD98059 (5 mM) pre-treatment for 1 hour, or appropriate controls. Cell free supernatants were collected and assayed by TNF-α ELISA kit (Genzyme, Cambridge, Mass.) or nitric oxide (NO) assay kit (Calbiochem, La Jolla, Calif.) in strict accordance with the manufacturer's instruction. The Bio-Rad protein assay (Bio-Rad Laboratories, Hercules, Calif.) was performed to measure total cellular protein from each of the cell groups under consideration just prior to quantification of cytokine release by ELISA or NO secretion by NO assay.

LDH Release Assay

Primary cultures of mouse cortical neurons were prepared as described above. Neuronal cells were seeded in 24-well tissue culture plates at 1×10⁵ cells/well for 48 hours and used as target cells for LDH release assay (Promega, Madison, Wis.). Neuronal cells or neuronal/microglial co-cultures (microglia, 5×10⁴ cells/well) were treated with phen (5 mM), Aβ₁₋₄₀ (1000 nM), Aβ₁₋₄₂ (1000 nM), control peptide (1000 nM), anti-CD45 antibody (1:200), phen/Aβ peptides, anti-CD45/phen/Aβ peptides, or appropriate controls. LDH release assay was carried out as described (Tan et al., 1999a) for 36 or 48 hours in neuronal cultures, microglial cultures or neuronal/microglial co-cultures. Total LDH release represents maximal lysis of target cells with 5% Triton X-100. All analyses were performed using SPSS for Windows, release 9.0.

Results

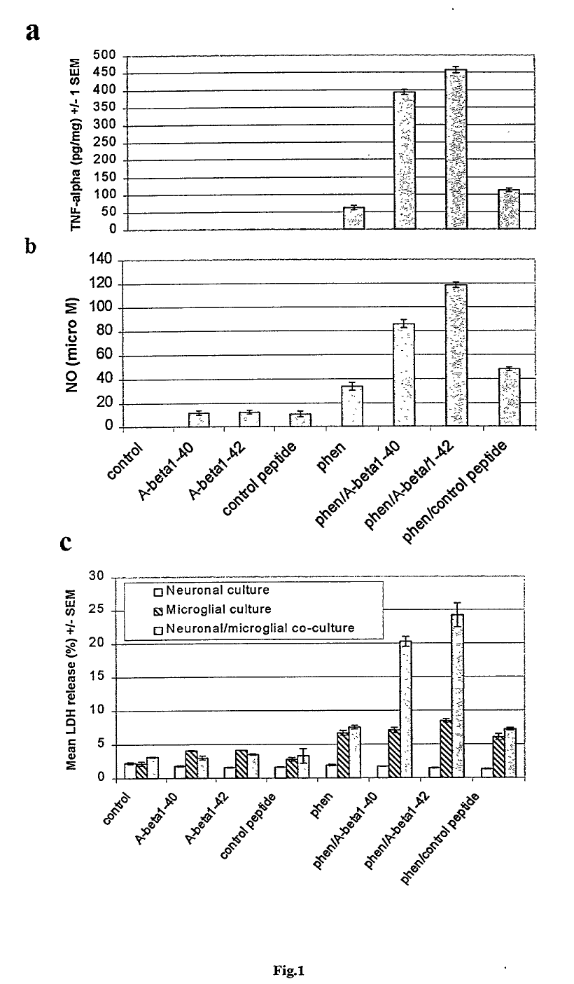

Co-treatment with phen and Aβ peptides results in synergistic microglial activation. It has been shown that a tyrosine phosphorylation cascade plays an important role in Aβ-induced microglial activation (McDonald et al., 1998; Combs et al., 1999). To test whether promotion of tyrosine phosphorylation could affect Aβ induced microglial activation, we co-incubated primary cultured microglial cells with phen, a specific tyrosine phosphatase inhibitor, and Aβ peptides for 12 hours. Microglial activation was measured by TNF-α and NO production, and neuronal cell injury in co-culture experiments. Data showed that phen synergistically enhanced Aβ-stimulated microglial activation (FIG. 1). This result led us to focus on stimulating microglial PTP activity in order to oppose Aβ-induced activation of these cells.

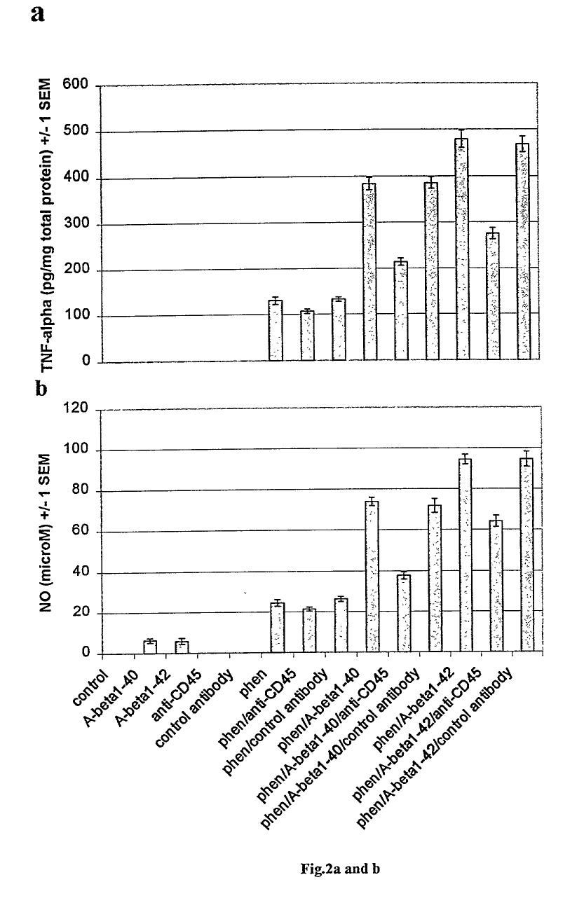

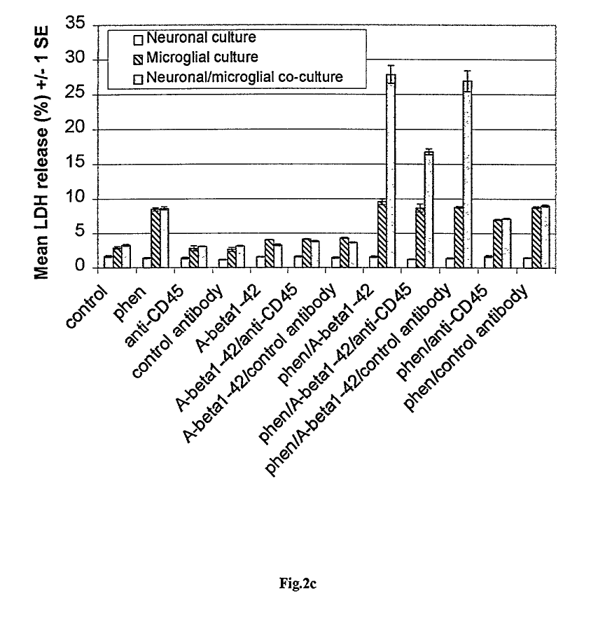

CD45 cross-linking significantly inhibits microglial activation induced by phen and Aβ peptides. It has been reported that CD45, a protein tyrosine phosphatase which is constitutively expressed in microglia (Karp et al., 1994), is markedly increased in microglia from AD frontal cortices (Masliah et al., 1991; Licastro et al., 1998). To examine the putative role of CD45 in microglial activation, we treated primary cultured microglial cells with monoclonal anti-CD45 antibody prior to stimulation with phen and Aβ peptides. Microglial activation, as evidenced by TNF-α and NO release following co-treatment of microglia with phen and Aβ peptides, was significantly inhibited by cross-linking CD45 (FIG. 2a and b). Hyper-stimulation of microglia commonly results in bystander cell injury, and we went on to evaluate whether cross-linking of CD45 might protect neuronal cells against injury from activated microglia (resulting from phen and Aβ peptide co-treatment). When activated microglia were co-cultured with primary cultured neuronal cells in the presence of anti-CD45 antibody, we observed that neuronal cells were significantly protected against injury induced by reactive microglia (FIG. 2c), further substantiating the role of CD45 in negative regulation of microglial activation.

Statistical Analysis

Data were analyzed using analysis of variance (ANOVA) followed by post hoc comparisons of means by Bonferroni's or Dunnett's T3 method, where Levene's test for homogeneity of variances was used to determine the appropriate method of post hoc comparison. In instances of single mean comparisons, t test for independent samples was used to assess significance. Alpha levels were set at 0.05 for each analysis.

### Example 2

### CD45 Cross-Linking Reduces p44/22 MAPK Activity

Materials And Methods

Western Immunoblotting

Murine primary culture microglia were plated in 6 well tissue-culture plates at a density of 8×10⁵ cells/well. These cells were incubated for 30 min with or without phen (5 mM) and Aβ peptides (1000 nM) in the presence or absence of anti-CD45 or control antibodies (1:200 dilution for each) or PD98059 (5 mM) pre-treatment for 1 hour, or appropriate controls. Immediately following culturing, microglia were washed in ice-cold phosphate buffered saline (PBS) 3×, scraped into ice-cold PBS, and lysed in an ice-cold lysis buffer containing 20 mM Tris (pH 7.5), 150 mM NaCl, 1 mM EDTA, 1 mM EGTA, 1% Triton X-100, 2.5 mM sodium pyrophosphate, 1 mM b-glycerolphosphate, 1 mM Na₃VO₄, 1 μg/mL leupeptin, and 1 mM PMSF. After incubation for 30 min. on ice, samples were centrifuged at high speed for 15 minutes, and supernatants were collected. Total protein content was estimated using the Bio-Rad protein assay. An aliquot corresponding to 50 μg of total protein of each sample was separated by SDS-PAGE and transferred electrophoretically to Hy-bond PVDF membranes. Non-specific antibody binding was blocked with 5% non-fat dry milk for 1 h at room temperature in TBS (20 mM Tris, 500 mM NaCl, pH 7.5). Membranes where first hybridized with a phospho-specific p44/22 MAPK antibody, stripped with b-Mercaptoethanol stripping solution (62.5 mM Tris-HCl, pH 6.8, 2% SDS, and 100 mM b-Mercaptoethanol), and then re-probed with an antibody that recognizes total p44/22 MAPK. Alternatively, membranes with identical samples were probed with either with a phospho-specific p44/22 MAPK antibody or with an antibody that recognizes total p44/22 MAPK. Immunoblotting was carried out with a primary antibody followed by an antimouse HRP-conjugated IgG secondary antibody as a tracer. The luminol reagent was used to develop the blots. Densiometric analysis was preformed for all blots using the Fluor-S Multimager™ with Quantity One™ software (Bio-Rad, Calif.).

TNF-a ELISA and Nitric Oxide Release Assay

For TNF-a Western blot, brains from 6-month-old transgenic mice were isolated under sterile conditions on ice and placed in ice-cold lysis buffer containing 20 mM Tris (pH 7.5), 150 mM NaCl, 1 mM EDTA, 1 mM EGTA, 1% Triton X-100, 2.5 mM sodium pyrophosphate, 1 mM b-Glycerolphosphate, 1 mM Na₃VO₄, 1 mg/mL Leupeptin and 1 mM PMSF. Brains were then sonicated on ice for approximately 3 min, let stand for 15 min at 4 degrees Centigrade, and centrifuged at 15,000 rpm for 15 min. Supernatants were then collected for protein assay and western immunoblotting as described above.

Immune Complex Kinase Assay

Primary culture microglial cells were seeded in 6-well tissue-culture plates at 8×10⁵ per well. Thirty minutes following co-treatment with phen and Aβ peptides in the presence or absence of anti-CD45 antibody or appropriate controls, microglial cells were lysed in ice-cold lysis buffer (as described above). Total cellular protein was quantified with the BioRad protein assay, and an aliquot of 100 mg of protein for each treatment condition was separated by SDS-PAGE. p44/22 MAPK activity was determined using the p44/22 MAP-Kinase Assay Kit (New England BioLabs, Beverly, Mass.) in strict accordance with the manufacturer's instruction. The phosphorylated form of the Elk 1 p44/22 MAPK fusion protein was visualized by Western immunoblotting (as described above) using a specific antibody for phosphorylated Elk 1 supplied with the kit.

Results

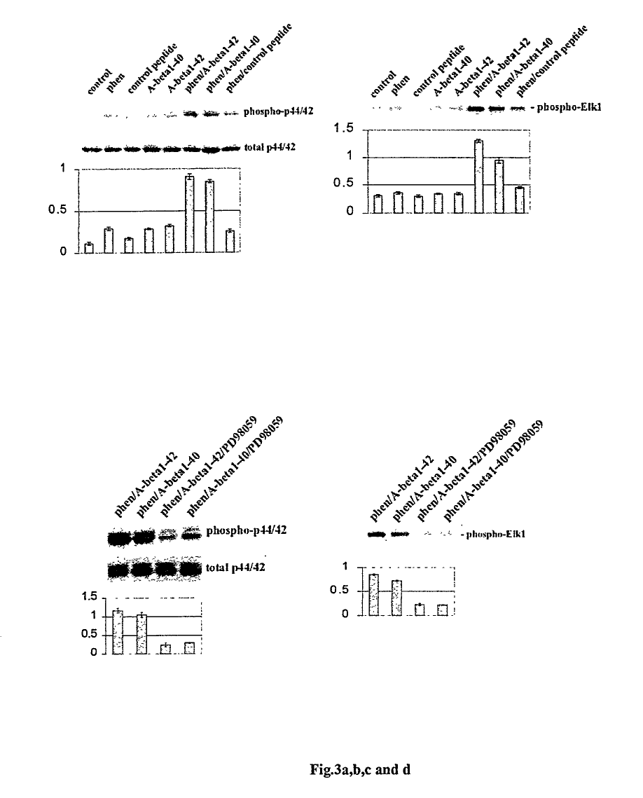

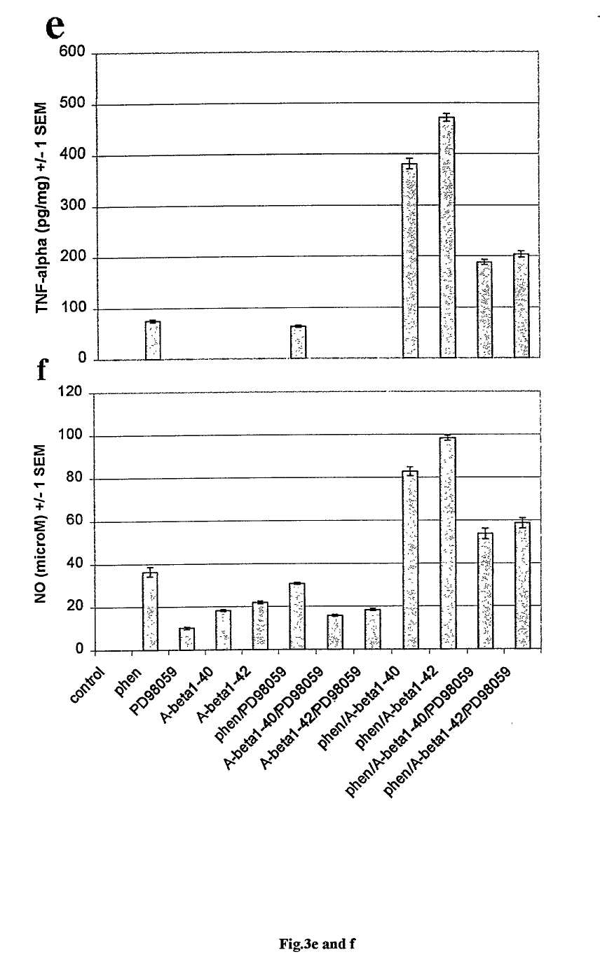

Cross-linking of CD45 suppresses microglial activation through a p44/22 MAPK-dependent pathway. Previous studies have shown that activation of mitogen activated protein kinase (MEK1/2) and downstream p44/22 MAPK is involved in TNF-α production in macrophages, monocytes and microglia following activation of these cells with a variety of stimuli, including lipopolysaccharide (LPS) and CD40 ligand (Hambleton et al., 1995; Suftles et al., 1999; Tan et al., 1999c). These data led us to ask whether the observed effect of CD45 cross-linking on opposing microglial activation might be mediated via activation of the MAPK module. Thus, we analyzed p44/22 MAPK phosphorylation status and activity in microglial cell lysates following co-treatment with phen and Aβ peptides or appropriate control conditions. Results showed that p44/22 MAPK phosphorylation and activity were both synergistically induced within 30 minutes after co-treatment with phen and Aβ₁₋₄₀ or Aβ₁₋₄₂ peptides (FIG. 3a and b). Furthermore, we observed that treatment of microglia with PD98059, a selective inhibitor of MEK1/2, results in significant reduction of phen and Aβ-mediated phosphorylation and activity of p44/22 MAPK (FIG. 3c and d).

To determine whether activation of p44/22 MAPK was responsible for TNF-α and NO production following co-treatment of microglia with phen and Aβ peptides, we treated microglia with PD98059 prior to stimulation with phen and Aβ peptides. Production of TNF-α and NO were markedly decreased compared to appropriate controls, within 12 hours post-treatment with PD98059 and phen/Aβ peptides (FIG. 3e and f). These data suggest that activation of p44/22 MAPK is crucial for microglial TNF-α and NO production following co-treatment of microglia with phen and Aβ peptides.

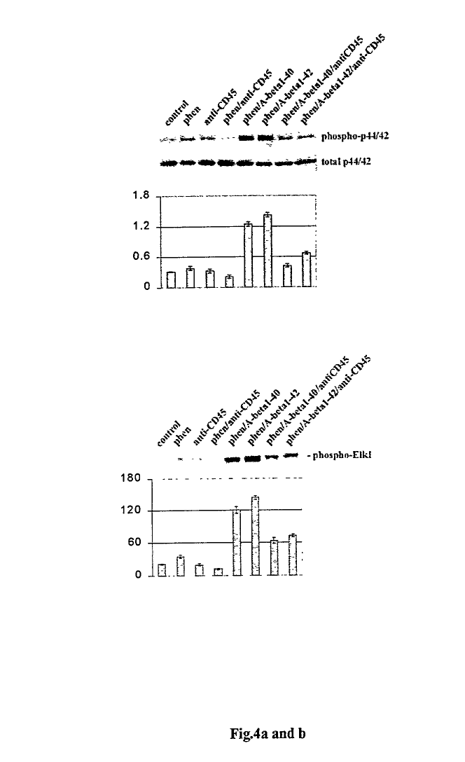

Having shown that cross-linking of CD45 opposed microglial activation, we wished to determine whether reduced p44/22 MAPK activity could be responsible for this effect. To investigate this possibility, microglial cells were co-incubated with anti-CD45 antibody, phen and Aβ peptides. Cell lysates were then analyzed for phosphorylated forms of p44/22 MAPK by Western immunoblotting. Results showed that cross-linking of CD45 significantly inhibited phosphorylation of p44/22 MAPK induced by phen and Aβ peptide co-treatment compared to controls (FIG. 4a). To determine if this effect could result in decreased MAPK activity, an immune complex kinase assay was performed. Results showed that cross-linking of CD45 markedly reduced p44/22 MAPK activity in phen and Aβ peptide co-treated cells (FIG. 4b), demonstrating the functionality of CD45 cross-linking on p44/22 MAPK activity.

Statistical Analysis

Data were analyzed using analysis of variance (ANOVA) followed by post-hoc comparisons of means by Bonferroni's or Dunnett's T3 method, where Levene's test for homogeneity of variances was used to determine the appropriate method of post-hoc comparison. In instances of single mean comparisons, t test for independent samples was used to assess significance. Alpha levels were set at 0.05 for each analysis.

### Example 3

### CD45 Negatively Regulates Microglial Activation

Materials And Methods

TNF-a ELISA and Nitric Oxide Release Assay

Both TNF-a ELISA and nitric oxide release assays were conducted as previously described in Example 1 using microglial cells from CD45-deficient and wild-type mice.

LDH Release Assay

The LDH release assay was performed as previously described in Example 1 using microglial cells from CD45-deficient and wild-type mice.

Western Immunoblotting

For TNF-a Western blot, brains from 6-month old CD45 deficient, Tg APP_{SW}, and Tg APP_{SW}/CD45 deficient mice were isolated under sterile conditions on ice and placed in ice-cold lysis buffer containing 20 mM Tris (pH 7.5), 150 mM NaCl, 1 mM EDTA, 1 mM EGTA, 1% Triton X-100, 2.5 mM sodium pyrophosphate, 1 mM b-Glycerolphosphate, 1 mM Na3VO4, 1 ug/mL Leupeptin, and 1 mM PMSF. Brains were then sonicated on ice for approximately 3 minutes, allowed to stand for 15 minutes at 4 C., and centrifuged at 15,000 rpm for 15 minutes. Supernatants were then collected for protein assay and Western immunoblotting as described above.

Results

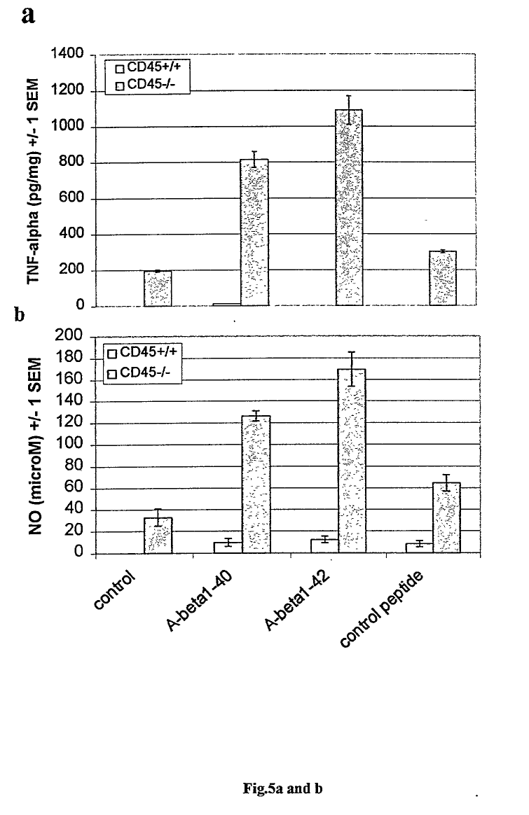

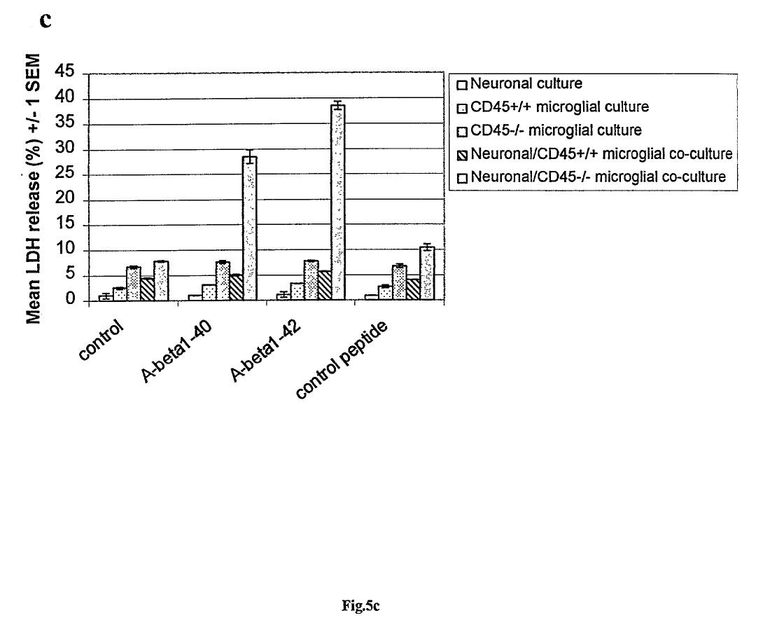

Stimulation of CD45-deficient microglia with Aβ peptides results in microglial activation. To further substantiate the role of CD45 in Aβ-mediated microglial activation, microglia were obtained from CD45 -deficient or wild-type mice and treated with either Aβ or control peptide for 12 hours. As described above, microglial activation was quantified by TNF-α and NO release. Results shown in FIGS. 5a and 5b indicate marked activation of CD45-deficient microglia compared to wild-type microglia following stimulation with Aβ peptides. Further, to determine whether CD45-deficient microglia in this scenario could cause neuronal cell injury, primary cultured cortical neurons and microglia were co-cultured, and morphologic examination (data not shown) and LDH assay were performed. Data showed that cortical neurons were markedly injured by Aβ-treated CD45-deficient microglia compared to wild-type cells (FIG. 5c). When taken together, these data show that CD45 is a negative regulator of Aβ-induced microglial activation.

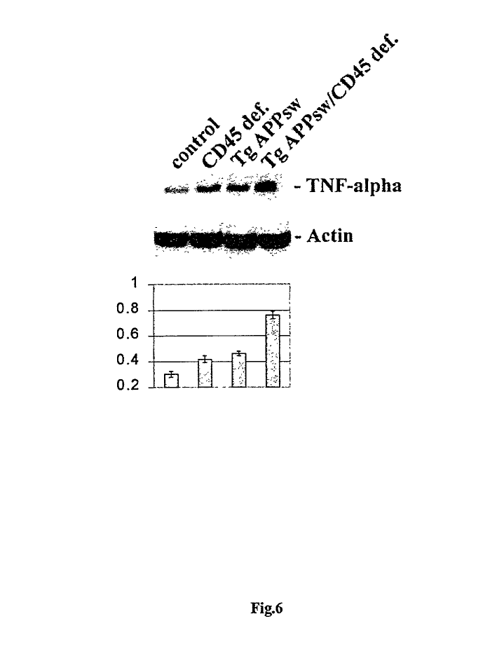

Tg APP_{SW} mice deficient for CD45 demonstrate marked TNF-α induction. To evaluate the possibility that CD45 might be a negative regulator of Aβ mediated microglial activation in vivo, we crossed transgenic Swedish APP over-expressing mice (Tg APP_{SW}) with mice deficient for CD45, and measured TNF-α production in the brains of these animals. Results showed a marked increase in TNF-α protein in brain homogenates from these mice compared to Tg APP_{SW} mice (FIG. 6). We were only able to detect CD45 on microglia in Tg APP_{SW} and control mice (data not shown), and CD45 has not been reported to be expressed by other CNS cells. Thus, these data suggest that microglial CD45 negatively regulates Aβ-induced microglial activation as evidenced by TNF-α production in vivo.

Statistical Analysis

Data were analyzed using analysis of variance (ANOVA) followed by post-hoc comparisons of means by Bonferroni's or Dunneft's T3 method, where Levene's test for homogeneity of variances was used to determine the appropriate method of post-hoc comparison. In instances of single mean comparisons, t test for independent samples was used to assess significance. Alpha levels were set at 0.05 for each analysis.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

There has thus been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended hereto.

In this respect, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### REFERENCES

1. McGeer, P. L. & McGeer, E. G. Inflammation in the brain in Alzheimer's disease: Implications for therapy. J. Leukocyte Biol. 65, 409-415 (1999).

2. McGeer, E. G. & McGeer, P. L. The importance of inflammatory mechanisms in Alzheimer's disease. Exp. Gerontol. 33, 371-378 (1998).

3. Rogers, J., et al. Inflammation and Alzheimer's disease pathogenesis. Neurobiol. Aging 17, 681-686 (1996).

4. Meda, L., et al. Activation of microglial cells by beta-amyloid and interferon-gamma. Nature 374, 647-650 (1995).

5. Barger, S. W. & Harmon, A. D. Microglial activation by Alzheimer amyloid precursor protein and modulation by apolipoprotein E. Nature 388, 878-881(1997).

6. Stewart, W. F., Kawas, C., Corrada, M. & Metter, E. J. Risk of Alzheimer's disease and duration of NSAID use. Neurology 48, 626-632 (1997).

7. Mackenzie, I. R. & Munoz, D. G. Nonsteroidal anti-inflammatory drug use and Alzheimer-type pathology in aging. Neurology 50, 986-990 (1998).

8. Lim, G. P. et al. Ibuprofen suppresses plaque pathology and inflammation in a transgenic mouse model for Alzheimer's disease. J. Neurosci. 20, 5709-5714 (2000).

9. Schenk, D. et al. Immunization with beta-amyloid attenuates Alzheimer-disease-like pathology in the PDAPP mouse. Nature 400, 173-177 (1999).

10. Bard, F. et al. Peripherally administered antibodies against amyloid beta-peptide enter the central nervous system and reduce pathology in a mouse model of Alzheimer disease. Nat. Med. 6, 916-919 (2000).

11. Morgan, D. et al. A beta peptide vaccination prevents memory loss in an animal model of Alzheimer's disease. Nature 408, 982-985 (2000).

12. Janus, C et al. A beta peptide immunization reduces behavioural impairment and plaques in a model of Alzheimer's disease. Nature 408, 979-982 (2000).

13. Bacskai, B. J., et al. Imaging of amyloid-beta deposits in brains of living mice permits direct observation of clearance of plaques with immunotherapy. Nat. Med. 7, 369-372 (2001).

14. Wyss-Coray, T. et al. TGF-betal promotes microglial amyloid-beta clearance and reduces plaque burden in transgenic mice. Nat. Med. 7, 612-618 (2001).

15. Grewal, I. S., et al. Requirement for CD40 ligand in costimulation induction, T cell activation, and experimental allergic encephalomyelitis. Science 273, 1864-1867 (1996).

16. Stuber, E., Strober, W. & Neurath, M. Blocking the CD40L-CD40 interaction in vivo specifically prevents the priming of T helper 1 cells through the inhibition of interleukin 12 secretion. J. Exp. Med. 183, 693-698 (1996).

17. Carson, M. J., Reilly, C. R., Sutcliffe, J. G. & Lo, D. Mature microglia resemble immature antigen-presenting cells. Glia 22, 72-85.

18. Tan, J. et al. Activation of microglial cells by the CD40 pathway: relevance to multiple sclerosis. J. Neuroimmunol. 97, 77-85 (1999).

19. Tan, J. et al. Microglial activation resulting from CD40-CD40L interaction after beta-amyloid stimulation. Science 286, 2352-2355 (1999).

20. Togo, T. et al. Expression of CD40 in the brain of Alzheimer's disease and other neurological diseases. Brain Res. 885, 117-121 (2000).

21. Irizarry, M. C., McNamara, M., Fedorchak, K., Hsiao, K. & Hyman, B. T. APPSw transgenic mice develop age-related A beta deposits and neuropil abnormalities, but no neuronal loss in CA1. J. Neuropathol. Exp. Neurol. 56, 965-73 (1997).

22. Nilsson, L. N., et al Alpha-1-antichymotrypsin promotes beta-sheet amyloid plaque deposition in a transgenic mouse model of Alzheimer's disease. J. Neurosci. 21, 1444-1451 (2001).

23. Harris-White, M. E., et al. Effects of transforming growth factor-beta (isoforms 1-3) on amyloid-beta deposition, inflammation, and cell targeting in organotypic hippocampal slice cultures. J. Neurosci. 18, 10366-10374 (1998).

24. Styren, S. D., Kamboh, M. I. & DeKosky, S. T. Expression of differential immune factors in temporal cortex and cerebellum: the role of alpha-1-antichymotrypsin, apolipoprotein E, and reactive glia in the progression of Alzheimer's disease. J. Comp. Neurol. 396, 511-520 (1998).

25. Rozemuller, J. M., Eikelenboom, P., Stam, F. C., Beyreuther, K. & Masters, C. L. A4 protein in Alzheimer's disease: primary and secondary cellular events in extracellular amyloid deposition. J. Neuropathol. Exp. Neurol. 48, 674-691 (1989).

26. Frackowiak, J., et al. Ultrastructure of the microglia that phagocytose amyloid and the microglia that produce beta-amyloid fibrils. Acta Neuropathol. (Berl.) 84, 225-233 (1992).

27. Wegiel, J., Wang, K. C., Tamawski, M. & Lach, B. Microglia cells are the driving force in fibrillar plaque formation, whereas astrocytes are a leading factor in plague degradation. Acta Neuropathol. (Berl.) 100, 356-364 (2000).

28. Lutgens, E., et al. Requirement for CD154 in the progression of atherosclerosis. Nat. Med. 5, 1313 1316 (1999).

29. Foy, T. M., Aruffo, A., Bajorath, J., Buhlmann, J. E. & Noelle, R. J. Immune regulation by CD40 and its ligand GP39. Annu. Rev. Immunol. 14, 591-617 (1996).

30. Xu, J. et al. Mice deficient for the CD40 ligand. Immunity 1, 423-431 (1994).

31. Hsiao, K. K., et al. Age-related CNS disorder and early death in transgenic FVB/N mice overexpressing Alzheimer amyloid precursor proteins. Neuron 15, 1203-1218.

32. Johnson-Wood, K., et al. Amyloid precursor protein processing and A beta42 deposition in a transgenic mouse model of Alzheimer disease. Proc. Natl. Acad. Sci. U S A 94, 1550-1555 (1997).

33. Burke and Olson, “Preparation of Clone Libraries in Yeast Artificial Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270 (1991).

34. Capecchi, “Altering the genome by homologous recombination” Science 244:1288-1292 (1989).

35. Davies et al., “Targeted alterations in yeast artificial chromosomes for interspecies gene transfer”, Nucleic Acids Research, Vol. 20, No. 11, pp. 2693-2698 (1992).

36. Dickinson et al., “High frequency gene targeting using insertional vectors”, Human Molecular Genetics, Vol. 2, No. 8, pp. 1299-1302 (1993).

37. Duff and Lincoln, “Insertion of a pathogenic mutation into a yeast artificial chromosome containing the human APP gene and expression in ES cells”, Research Advances in Alzheimer's Disease and Related Disorders, 1995.

38. Huxley et al., “The human HPRT gene on a yeast artificial chromosome is functional when transferred to mouse cells by cell fusion”, Genomics, 9:742-750 (1991).

39. Jakobovits et al., “Germ-line transmission and expression of a human-derived yeast artificial chromosome”, Nature, Vol. 362, pp. 255-261 (1993).

40. Lamb et al., “Introduction and expression of the 400 kilobase precursor amyloid protein gene in transgenic mice”, Nature Genetics, Vol. 5, pp. 22-29 (1993).

41. Pearson and Choi, “Expression of the human b-amyloid precursor protein gene from a yeast artificial chromosome in transgenic mice.” Proc. Natl. Acad. Sci. USA, 1993. 90:10578-82.

42. Rothstein, “Targeting, disruption, replacement, and allele rescue: integrative DNA transformation in yeast” in Methods in Enzymology, Vol. 194, “Guide to Yeast Genetics and Molecular Biology”, eds. C. Guthrie and G. Fink, Academic Press, Inc., Chap. 19, pp. 281-301 (1991).

43. Schedl et al., “A yeast artificial chromosome covering the tyrosinase gene confers copy number-dependent expression in transgenic mice”, Nature, Vol. 362, pp. 258-261 (1993).

44. Strauss et al., “Germ line transmission of a yeast artificial chromosome spanning the murine al (1) collagen locus”, Science, Vol. 259, pp. 1904-1907 (1993).

45. Gilboa, E, Eglitis, M A, Kantoff, P W, Anderson, W F: Transfer and expression of cloned genes using retroviral vectors. BioTechniques 4(6):504-512, 1986.

46. Cregg J M, Vedvick T S, Raschke W C: Recent Advances in the Expression of Foreign Genes in Pichia pastoris, BioTechnology 11:905-910, 1993.

47. Culver, 1998. Site-Directed recombination for repair of mutations in the human ADA gene. (Abstract) Antisense DNA & RNA based therapeutics, February, 1998, Coronado, Calif.

48. Huston et al, 1991 “Protein engineering of single-chain Fv analogs and fusion proteins” in Methods in Enzymology (J J Langone, ed.; Academic Press, New York, N.Y.) 203:46-88.

49. Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99.

50. Mernaugh and Mernaugh, 1995 “An overview of phage-displayed recombinant antibodies” in Molecular Methods In Plant Pathology (R P Singh and U S Singh, eds.; CRC Press Inc., Boca Raton, Fla.) pp. 359-365.

51. Beard C M, Waring S C, O'Brien P C, Kurland L T, Kokmen E (1998). Nonsteroidal anti-inflammatory drug use and Alzheimer's disease: a case control study in Rochester, Minn., 1980 through 1984. Mayo Clin Proc 73-.951-955.

52. Chao C C, Hu S, Molitor T W, Shaskan E D, Peterson P K (1992) Activated microglia mediate neuronal cell injury via nitric oxide mechanism. J. Immunol. 149:2736-2741.

53. Combs C K, Johnson D E, Cannady S B, Lehman T M, Landreth G E (1999) Identification of microglial signal transduction pathways mediating a neurotixic response to amyloidogenic fragments of beta-amyloid and prion proteins. J. Neurosci. 19:928-939.

54. Friedman W J, Greene A (1995) Neurotrophin signaling via Trks and p75. Exp. Cell Res. 253:131-142.

55. Gong C X, Shaikh S, Wang J Z, Zaidi T, Grundke-lqbal K, lqbal K (1995) Phosphatase activity toward abnormally phophorylated tau: decrease in Alzheimer disease brain. J. Neurochem. 65: 732-738.

56. Hambleton J, McMahon M, DeFrarico A L (1995) Activation of Raf-1 and mitogen activated protein kinase in murine macrophages partially mimics lipopolysaccharide-inducing events. J. Exp. Med. 182:147-154.

57. Hsiao K K, Chapman P, Nilsen S, Eckman C, Harigaya Y, Younkin S, Yang F, Cole G (1996) Correlative memory deficits, Abeta elevation, and amyloid plaques in transgenic mice. Science 274:99-102.

58. Ikeda T, Yamamoto K, Takahashi K, Yamada M (1991) Immune systemassociated antigens on the surface of peripheral blood lymphocytes in patients with Alzheimer's disease. Acta Psychiatr. Scand. 83:444-448.

59. Justement L B (1996) The role of CD45 in signal transduction. Adv. Immunol. 66:1-65.

60. Karp H L, Tillotson M L, Sorita J, Reich C, Wood J G (1994) Microglial tyrosine phosphorylation systems in normal and degenerating brain. Glia 11:284-290.

61. Lazaar A L, Amrani Y, Hsu J, Paneftieri R A Jr, Fanslow W C, Albeida S M, Pure E (1998) CD40-mediated signal transduction in human airway smooth muscle. J. Immunol. 161:3120-3127.

62. Licastro F, Mallory M, Hansen L A, Masliah E (1998) Increased level of alpha 1-antichymotrypin in brains of patients with Alzheimer's disease correlate with activated astrocytes and are affected by APOE 4 genotype. J. Neuroimmunol. 88:105-110.

63. Loh R K, Jabara H H, Ren C L, Fu S M, Vercelli D, Geha R S (1995) Role of protein tyrosine kinases and phosphatases in isotype switching: crosslinking CD45 to CD40 inhibits IgE isotype switching in human B cells. Immunology Letters 45:99-106.

64. Masliah E, Mallory M, Hansen L, Alford M, Albright T, Terry R, Shapiro P, Sundsmo M, Saitoh T (1991) Immunoreactivity of CD45, a protein phophotyrosine phosphatase, in Alzheimer's disease. Acta Neuropathol. (Berl) 83:12-20.

65. Maxwell J R, Campbell J D, Kim C H, Vella A T (1999) CD40 activation boosts T cell immunity in vivo by enhancing T cell clonal expansion and delaying peripheral T cell deletion. J. Immunol. 162:2024-2034.

66. McDonald D R, Bamberger M E, Combs C K, Landreth G E (1998) beta-Amyloid fibrils activate parallel mitogen-activated protein kinase pathways in microglia and THP1 monocytes. J. Neurosci. 18:4451-4460.

67. Pei J J, Gong C X, lqbal K, Grundke-lqbal 1, Wu Q L, Winblad B, Cowburn R F (1998) Subcellular distribution of protein phosphatases and abnormally phosphorylated tau in the temporal cortex from Alzheimer's disease and control brains. J. Neural. Transm. 105:69-83.

68. Rich J B, Rasmusson D X, Folstein M F, Carson K A, Kawas C, Brandt J (1995) Nonsteroidal anti-inflammatory drugs in Alzheimer's disease. Neurology 45:51-55.

69. Suttles J, Milhom D M, Miller R W, Poe J C, Wahl L M, Stout R D (1999) CD40 signaling of monocyte inflammatory cytokine synthesis through an ERK1/2dependent pathway. A target of interleukin anti-inflammatory action. J. Biol. Chem. 26:5835-5842.

70. Tan J, Town T, Saxe M, Paris D, Wu Y, Mullan M (1999c) Ligation of microglial CD40 results in p44/22 mitogen-activated protein kinase dependent TNF-A production that is opposed by TGF-b1 and IL-10. J. Immunol. 163:6614-6621.

71. Yang Y, Wilson J M (1996) CD40 ligand-dependent T cell activation: requirement of B7-CD28 signaling through CD40. Science 273:1862-1864.

What is claimed is:

1. A method of inhibiting the negative effects of beta-amyloid in the brain of an animal comprising administering an effective amount of a compound that modulates CD45 activity.

2. The method of claim 1 in which the negative effects are selected from the group consisting of release of TNF-α, NO production, glutamate release, and neuronal cell injury, or combinations thereof.

3. The method of claim 1 in which the negative effects are selected from the group consisting of chronic activation of inflammatory cells, free radical mediated damage to cells or tissues, neuronal apoptosis, neuronal death, and release of inflammatory molecules, or combinations thereof.

4. The method of claim 3 in which the inflammatory cells are selected from the group consisting of microglia, macrophages, cells of monocytes, and astrocytes, alone or in combination.

5. The method of claim 1 in which the animal is a mammal.

6. The method of claim 1 in which the animal is a human.

7. The method of claim 1 in which the compound is an agonist that stimulates CD45 activity.

8. The method of claim 1 in which the compound is an antibody that stimulates CD45 activity.

9. The method of claim 1 in which the compound is an agonist that opposes CD40 ligation or the effects thereof.

10. The method of claim 7 in which the CD45 is expressed by immune cells.

11. The method of claim 10 in which the immune cells include brain microglia.

12. The method of claim 1 in which the animal exhibits features of a neurodegenerative disease.

13. The method of claim 12 in which the neurodegenerative disease is selected from the group consisting of Alzheimer's disease, Lewy body disease, tauopathies, Parkinson's disease, cerebrovascular disease, and prion disease, or combinations thereof.

14. The method of claim 1 in which the negative effects of beta-amyloid are selected from the group consisting of beta-amyloid deposition, increased beta-amyloid levels, increased formation of soluble or insoluble aggregates of beta-amyloid, neuronal injury, and tau pathology, or combinations thereof.

15. The method of claim 1 in which CD45 activity is measured by phosphorylation of a CD45 substrate or production of an inorganic phosphate.

16. The method of claim 15 in which the substrate is selected from the group consisting of src, a member of the src family, mitogen activated protein kinases, the c-Jun N-terminal kinase pathway, the c-Jun N-terminal kinase activating kinase/signal transducers, and activators of transcription pathway, or combinations thereof.

17. An assay for compounds that inhibit the negative effects of beta-amyloid comprising (a) contacting immune cells with a predetermined amount of one or more test compounds in the presence of one or more beta-amyloid peptides, (b) monitoring the amount of one or more inflammatory molecules released by the immune cells, and (c) comparing the amount found in step (b) with (i) another amount found in the absence of the one or more test compounds or (ii) using a different predetermined amount of the one or more test compounds.

18. The assay of claim 17 in which the one or more inflammatory molecules are selected from the group consisting of TNF-alpha, IL-1b, IL-6, IL-12, or TNF-gamma.

19. The assay of claim 17 in which the one or more inflammatory molecules are selected from the group consisting of NO, glutamate, a free radical species, superoxide dismutase, and nitric oxide synthase.

20. The assay of claim 17 in which the immune cells comprise human immune cells.

21. The assay of claim 20 in which the immune cells are selected from the group consisting of microglia, T cells, granulocytes, macrophages, and cells belonging to the monocyte lineage.

22. The assay of claim 17 in which the immune cells are deficient in CD45.

23. An assay for compounds that inhibit the negative effects of beta-amyloid comprising (a) contacting beta-amyloid overproducing immune cells with a predetermined amount of one or more test compounds in the presence of one or more stimulatory molecules, (b) monitoring the amount of one or more metabolite of amyloid precursor protein (APP) produced by the beta-amyloid overproducing immune cells, and (c) comparing the amount found in step (b) with (i) another amount found in the absence of the one or more test compounds or (ii) using a different predetermined amount of the one or more test compounds.

24. The assay of claim 23 in which the beta-amyloid overproducing cells are deficient in CD45.

25. An assay for identifying compounds that bind to CD45 comprising contacting CD45 with a predetermined amount of one or more test compounds in the presence of a substrate of CD45 and measuring CD45 activity.

26. The method of claim 25 in which CD45 activity is measured by phosphorylation of a CD45 substrate or production of an inorganic phosphate.

27. The method of claim 26 in which the substrate is src, a member of the src family, mitogen activated protein kinases, the c-Jun N-terminal kinase pathway, the c-Jun N-terminal kinase activating kinase/signal transducers and activators of transcription pathway, or combinations thereof.

28. The assay of claim 25 in which CD45 activity is measured in the presence of the one or more test compounds is compared to another amount of phosphorylation measured in the absence of the one or more test compounds or using a different predetermined amount of the one or more test compounds.

29. An in vivo assay for determining the biological activity of a test compound comprising (a) crossing a first animal model of Alzheimer's disease with a second animal deficient in CD45, (b) administering a predetermined amount of one or more test compounds to the offspring of step (a), and (c) determining the negative effects of beta-amyloid in the brain of the offspring.

30. The assay of claim 26 in which the negative effects of beta-amyloid in the brain are qualitatively assessed or quantitatively measured or both.

31. An assay comprising (a) obtaining or generating an animal model of neurodegenerative disease, (b) administering a molecule that modulates CD45 activity to the animal model, and (c) measuring negative effects of neurodegeneration in the animal model.

32. The method of claim 31 where the neurodegenerative disease is Alzheimer's disease.

33. The method of claim 31 where the neurodegenerative disease is selected from the group consisting of Parkinson's disease, Lewy body dementia, traumatic brain injury, tauopathies, prion disease, and vascular dementia, or combinations thereof.

34. The method of claim 31 where the molecule that modulates CD45 activity is an antibody that stimulates CD45 activity.

35. The method of claim 31 where the molecule that modulates CD45 activity is a pharmacological agent.
